(19) United States
(12) Patent Application Publication
(10) Pub. No.: US 2022/0152382 A1
(43) Pub. Date: May 19, 2022

(54) Catheter Device for Implanting a Medical Device

(71) Applicant: BIOTRONIK SE & Co. KG, Berlin (DE)

(72) Inventors: Nicholas Devich, Tualatin, OR (US); Eric Austin, Portland, OR (US); Hannes Kraetschmer, West Linn, OR (US)

(73) Assignee: BIOTRONIK SE & Co. KG, Berlin (DE)

(21) Appl. No.: 17/437,718

(22) PCT Filed: Mar. 11, 2020
(86) PCT No.: PCT/EP2020/056498

§ 371 Date: Sep. 9, 2021

Related U.S. Application Data
(60) Provisional application No. 62/818,774, filed on Mar. 15, 2019.

(51) Int. Cl. A61N 1/05; A61M 25/01

(52) U.S. Cl. CPC A61N 1/057; A61B 17/3468; A61M 25/0136; A61M 25/0147

(57) ABSTRACT

A catheter device for implanting a medical device comprises a steerable catheter, and a delivery catheter extending through the steerable catheter and being configured to interact with the medical device for implanting the medical device at an implantation site within a patient, wherein the delivery catheter is axially movable with respect to the steerable catheter. The steerable catheter comprises a first steering articulation zone and a second steering articulation zone arranged distally with respect to the first steering articulation zone, wherein the steerable catheter is steerable by a deflection in the first steering articulation zone and the second steering articulation zone.

### CROSS-REFERENCE TO RELATED APPLICATIONS

This application is the United States national phase under 35 U.S.C. § 371 of PCT International Patent Application No. PCT/EP2020/056498, filed on Mar. 11, 2020, which claims the benefit of U.S. Patent Application No. 62/818,774, filed on Mar. 15, 2019, the disclosures of which are hereby incorporated by reference herein in their entireties.

### TECHNICAL FIELD

The present disclosure relates to a catheter device for implanting a medical device according to the preamble of claim 1.

### BACKGROUND

In particular, the disclosure relates to a catheter design, in particular for septal and/or apical implantation of a leadless pacemaker in the right ventricle of the heart.

A catheter device of this kind comprises a steerable catheter, and a delivery catheter extending through the steerable catheter and being configured to interact with the medical device for implanting the medical device at an implantation site within a patient, wherein the delivery catheter is axially movable with respect to the steerable catheter.

Implantation of a medical device, also called implant in the following, such as a leadless pacemaker requires proper steering, positioning, and delivery of the medical device to a specific location of, e.g., the heart. The normal implantation procedure to implant, for example, a leadless pacemaker within the right ventricle of the heart utilizes access to the femoral vein of a patient. An introducer is installed that traverses from an incision site up into the atrium of the heart. Once the introducer is in place, a catheter device is required that can safely house the implant and its fixation mechanism and safely protect it from harmful interactions with either the patient's anatomy or the introducer. The catheter device is then inserted through the introducer until it exits the introducer in the atrium. Once a distal end of the catheter device is no longer contained within the introducer, it must navigate across the tricuspid valve and into the right ventricle. Once the distal end of the catheter device is in the right ventricle, the implant is deployed into the heart wall tissue at for example the apex or the septum of the right ventricle, wherein it may be clinically preferred to implant the medical device within the septum because the heart wall tissue is thicker in that location and there is less risk associated with implantation there. However, it may be more challenging to position the catheter device for deployment of the medical device in the septum and to allow for the medical device's fixation mechanism to reliably engage with the trabeculae in that area.

Once the medical device has been deployed within the right ventricle, electrical measurements and fixation assessments are generally performed by the user to confirm that an implantation site is suitable prior to releasing the medical device. If any electrical or fixation testing results are shown to be unacceptable, the implant is recaptured, repositioned, and implanted at a new location. Once the electrical and fixation testing measurements are found to be acceptable, the user shall then release the implant from the catheter. After the implant has successfully been released, the catheter device is removed from the patient anatomy.

There is a general desire for a catheter device that allows a user the means to navigate easily and reliably to an implantation site, for example across the tricuspid valve towards the septum or the apex within the right ventricle. Once navigated to a chosen implantation site, the user shall safely and reliably be able to deploy the medical device into that location. If it is determined through measurements that an implantation site is not acceptable, there beneficially should be a possibility to recapture the medical device and to reposition and release it within a new location.

U.S. Publication No. 2018/0280686 A1 discloses a delivery system for delivering an implantable leadless pacing device. Within the delivery system an intermediate tubular member may be advanced across the tricuspid valve and into the right ventricle. An outer tubular member of the delivery system may be torqued in a first direction to guide a distal holding section along the ventricular septum. The distal tip of the distal holding section may be releasably secured to a tissue. After securing the distal tip of the distal holding section, the outer tubular member may be torqued in a second direction opposite to the first direction and the implantable leadless pacing device incrementally deployed.

The present disclosure is directed toward overcoming one or more of the above-mentioned problems, though not necessarily limited to embodiments that do.

### SUMMARY

It is an object to provide a catheter device that allows a user to safely and reliably steer the catheter device for implantation of a medical device, and navigate it across tight structures, e.g. the tricuspid valve between the atrium and the right ventricle.

At least this object is achieved by means of a catheter device comprising the features of claim 1.

Accordingly, the steerable catheter comprises a first steering articulation zone and a second steering articulation zone arranged distally with respect to the first steering articulation zone, wherein the steerable catheter is steerable by a deflection in the first steering articulation zone and the second steering articulation zone.

In particular, a steering of the catheter device may be achieved solely by providing a steering function on the steerable catheter. The delivery catheter in contrast may, in one embodiment, be non-steerable and hence cannot particularly deflect in order to steer the catheter device, in particular through tight structures such as the tricuspid valve for example for implanting a medical device in the shape of a leadless pacemaker.

The steerable catheter comprises (at least) two steering articulation zones which may cause a deflection on the steerable catheter and hence provide for a steering function of the catheter device. A first steering articulation zone is arranged proximally with respect to a second steering articulation zone, such that a steering function may be provided by means of the steerable catheter within two zones which are axially displaced with respect to one another.

In particular, the second steering articulation zone may be formed in the vicinity of the distal end of the steerable catheter. The first steering articulation zone is arranged proximally of the second steering articulation zone and hence is arranged closer to a proximal end of the steerable catheter.

The steerable catheter may be actively steered or passively steered using the first steering articulation zone and the second steering articulation zone.

In one embodiment, the catheter device comprises a steering device which is actuatable for actively causing a deflection in at least one of the first steering articulation zone and the second steering articulation zone. The steering device may in particular comprise a pull wire which may be pulled in order to cause, actively, a deflection at an associated steering articulation zone. A pull wire of this kind may for example be guided within a guide lumen within the steerable catheter, for example formed within a catheter wall of the steerable catheter, such that the pull wire may extend from the associated steering articulation zone towards a proximal end of the steerable catheter in order to be actuated at the proximal end of the steerable catheter to cause a deflection at the respective steering articulation zone.

The steering articulation zone may be configured to be articulatable in a single direction or in two or more directions, i.e. within a plane perpendicular to the longitudinal axis of extension of the catheter device. If the steering articulation zone is articulatable in a single direction, a single steering device in the shape of a pull wire may be provided to cause a deflection at the steering articulation zone. If the steering articulation zone is articulatable in two directions, two steering devices in the shape of pull wires may be provided to cause a deflection in the two directions.

In one embodiment, both steering articulation zones are steerable using steering devices such that both steering articulation zones are actively deflectable.

It however also is conceivable that only one steering articulation zone is actively deflectable using a steering device, whereas the other steering articulation zone is passively deflectable as shall be explained further below.

In one embodiment, the catheter device comprises a handle device arranged at a proximal end of the steerable catheter and comprising at least one actuation member configured to control said steering device for causing a deflection in the at least one of the first steering articulation zone and the second steering articulation zone. The steering device hence can be controlled from the handle device, which is arranged proximally on the catheter device and hence remains outside of the patient when introducing the catheter device into the patient for implanting a medical device at an implantation site for example in the patient's heart.

If multiple steering devices are present in the shape of multiple pull wires, the handle device may comprise multiple actuation members, each actuation member serving to actuate one of the steering devices in the shape of the pull wires. If for example both steering articulation zones are actively deflectable using one steering device each, the handle device may comprise two actuation members, one actuation member being designed to actuate the steering device associated with the first steering articulation zone, and a second actuation member being designed to actuate the steering device associated with the second steering articulation zone.

The at least one actuation member of the handle device may for example have the shape of a rotatable steering knob.

Steering devices associated with the different steering articulation zones may in particular be independently controllable using the associated actuation members.

In another embodiment, a single actuation member for example in the shape of a steering knob may be provided in order to cause a deflection at both steering articulation zones, wherein for an independent control of the deflection the actuation member for example can be toggled for controlling one of the steering articulation zones or the other.

The handle device may in particular be designed such that the at least one actuation member remains stationary if a user releases the handle device, such that a deflection as controlled by the at least one actuation member is maintained.

In another embodiment, at least one of the first steering articulation zone and the second steering articulation zone may be not actively steerable, but may be passively steerable. For this, at least one of the first steering articulation zone and the second steering articulation zone may be formed by a shape-set curve. This is to be understood in that the first steering articulation zone and/or the second steering articulation zone is formed such that it, in a relaxed state, assumes a predefined curvature and bending differing from a straight extension, such that the respective steering articulation zone transitions to its predefined curvature if no other forces act onto the respective steering articulation zone.

Herein, a steering may be achieved using the respective steering articulation zone by for example using an introducer through which the catheter device is introduced towards an implantation site, for example towards the patient's heart. The introducer may form a cylindrical, straight hollow channel through which the catheter device is introduced. If a steering articulation zone is formed by a shape-set curve, it is forced to assume a straight shape when introduced through the introducer. Once the respective steering articulation zone has passed the introducer and exits from the introducer at its distal end, the steering articulation zone however reverts to its pre-shaped, curved shape, which may be used to steer the catheter device towards an implantation site, for example through a tight structure such as a tricuspid valve to enter the right ventricle within the patient's heart.

In one embodiment, one of the steering articulation zones is formed by a shape-set curve and is not actively steerable. It however also is conceivable that both steering articulation zones are formed by shape-set curves and are not actively steerable.

In yet another embodiment, it is conceivable to form the steering articulation zones by shape-set curves and in addition provide for an active steering function in the steering articulation zones by providing steering devices for actively deflecting the steering articulation zones.

The steerable catheter as a whole or at least in sections forming the steering articulation zones may for example be formed from a thermoplastic elastomer such as a Polyether block amide material and may comprise a stainless steel braided wire, nitinol reinforcement wires and/or a PTFE liner.

In one embodiment, the first steering articulation zone comprises a first length and/or a first bending radius, whereas the second steering articulation zone comprises a second length and/or a second bending radius. The first length herein is different than the second length, and/or the first bending radius is different than the second bending radius. The length is measured along the axial direction of the catheter device. The bending radius defines the curvature in the respective steering articulation zone.

In particular, the first steering articulation zone may have a longer length than the second steering articulation zone. In this way it can be achieved that a deflection in the first steering articulation zone takes place at a larger bending radius than a deflection at the second, distal steering articulation zone. In particular within the second steering articulation zone a deflection at a relatively tight radius may be achieved, whereas a deflection in the first steering articulation zone takes place at a substantially larger bending radius. Hence, by deflecting the steerable catheter distally at the second steering articulation zone a steering across tight structures may be achieved in that the steerable catheter may be navigated around sharp curves and through tight structures such as the tricuspid valve using in particular a tight deflection at the second steering articulation zone.

In one embodiment, the steerable catheter comprises a straight shaft section arranged proximally of the first steering articulation zone. The straight shaft section does not have a preshaped curvature, but may be flexible such that it may follow and adapt if the catheter device is introduced into a patient towards an implantation site.

The straight shaft section may have an increased rigidity in comparison to for example the first steering articulation zone adjoining the straight shaft section. The straight shaft section may be formed from a different material, for this purpose, in comparison to the first steering articulation zone and/or the second steering articulation zone.

In one embodiment, a protective cup is arranged on the steerable catheter for releasing the medical device. During implantation of a medical device such as a leadless pacemaker the medical device is received within the protective cup, which serves to sheathe the medical device such that it is covered towards the outside, enclosing in particular also fixation devices such as fixation tines arranged at a distal end of a body of the medical device and protruding from the body towards the outside. Hence, by sheathing the medical device a safe delivery may be ensured, without a risk of the medical device prematurely (i.e. prior to reaching an implantation site) engaging or otherwise interfering with tissue.

For the delivery of the medical device towards an implantation site the medical device is operatively connected to the delivery catheter, which is movable within the steerable catheter. Hence, by moving the delivery catheter with respect to the steerable catheter the medical device, when an implantation site is reached, may be deployed from the protective cup arranged on the steerable catheter, such that the medical device may engage with tissue and hence may be placed and secured at the implantation site.

In one embodiment, the catheter device comprises a mandrel guided in an inner lumen of the delivery catheter, the mandrel being movable with respect to the delivery catheter, and an adapter piece connected to the mandrel. The adapter piece, by moving the mandrel with respect to the delivery catheter, is displaceable between a first position in which the adapter piece is received within the inner lumen of the delivery catheter and a second position in which the adapter piece is arranged outside of the inner lumen of the delivery catheter. The catheter device furthermore comprises a tethering member comprising a tether portion having a first end at which the tethering member is connected to the adapter piece, wherein the tethering member further comprises a positive-locking member adjoining the tether portion at a distance from the first end. The positive-locking member in a connection state is received on the adapter piece and is locked with respect to the adapter piece while the adapter piece is in the first position. The positive-locking member is releasable from the adapter piece by displacing the adapter piece from the first position towards the second position.

Hence, a tethering mechanism is provided on the delivery catheter serving to securely hold the medical device on the delivery catheter during the implantation procedure and to easily release the medical device from the delivery catheter once an implantation site has been reached. The adapter piece serves to control the establishment and release of a connection of a medical device to the catheter device. For this, the tethering member is connected to the adapter piece, the tethering member at a first end being arranged on the adapter piece and carrying, at a distance from the first end, a positive-locking member which releasably may be connected to the adapter piece. In a connection state, in which the positive-locking member is received on the adapter piece, the tethering member by means of its tether portion hence forms a loop, which allows for a connection of a medical device to the catheter device, for example in that the tether portion of the tethering member extends through an opening of a connection member of the medical device such that by means of the tether portion the medical device is securely held on the catheter device.

The positive-locking member in the connection state is received on the adapter piece and is locked with respect to the adapter piece while the adapter piece is in the first position and hence is received within the inner lumen of the delivery catheter. The positive-locking member in the connection state is held on the adapter piece in a positive-locking manner such that the fixation of the medical device to the catheter device is locked.

For releasing the medical device from the catheter device, the adapter piece may be moved from its first position towards the second position and hence may slide out of the delivery catheter. When the adapter piece has exited the delivery catheter, the locking of the positive-locking member with respect to the adapter piece is released, such that the positive-locking member may disengage from the adapter piece and the loop previously formed by the tethering member is opened. The medical device hence may be disconnected from the catheter device.

The releasing of the medical device from the catheter device hence is controlled by moving the adapter piece with respect to the delivery catheter. The moving of the adapter piece takes place by actuating a mandrel, the mandrel being connected to the adapter piece such that the adapter piece may be displaced from its first position within the delivery catheter towards the second position in which the adapter piece is placed outside of the delivery catheter.

In one embodiment, the positive-locking member is arranged at a second end of the tether portion opposite the first end at which the tether portion is connected to the adapter piece. The tether portion in between the first end and the second end hence forms a loop if the positive-locking member is received on and is locked with respect to the adapter piece, such that by means of the tether portion a secure connection of a medical device to the catheter device may be established.

In one embodiment, the positive-locking member comprises a first diameter, when measured along a plane perpendicular to a longitudinal direction of extension of the tethering member, larger than a second diameter of the tether portion. The positive-locking member may for example have a spherical shape and hence provides for a widened diameter at the second end of the tether portion. The positive-locking member, in its connection state, is received on the adapter piece such that it is held on the adapter piece in a positive-locking fashion, the positive-locking member interacting with a suitable counter-portion of the adapter piece such that the positive-locking member cannot be released from the adapter piece, at least not without moving the adapter piece from its first position in which it is received within the delivery catheter to the second position in which it is placed outside of the delivery catheter.

In one embodiment, the adapter piece comprises a body and a retainer groove formed on the body, wherein the tether portion in the connection state of the positive-locking member is received in the retainer groove. The positive-locking member herein may be received in a recess formed on the body of the adapter piece, the recess adjoining the retainer groove. The retainer groove has a width such that the tether portion may be received within the retainer groove, but the diameter of the positive-locking member exceeds the width of the retainer groove such that the positive-locking member cannot be pulled through the retainer groove, hence providing for a fixation of the positive-locking member on the adapter piece.

The retainer groove may extend along a longitudinal direction along which the delivery catheter generally extends and along which the adapter piece is movable within the inner lumen of the delivery catheter. The retainer groove as well as the recess adjoining the retainer groove herein may be opened towards a lateral side, i.e. in a direction transverse to the longitudinal direction defined by the delivery catheter. Hence, the positive-locking member as well as the tether portion may be removed from the adapter piece along the transverse direction by disengaging the positive-locking member from the recess and by removing the tether portion from the retainer groove. However, as the adapter piece in its first position is received within the inner lumen of the delivery catheter, a disengagement of the positive-locking member from the recess is blocked by the delivery catheter, such that the connection of the positive-locking element to the adapter piece is locked. Only upon moving the adapter piece to exit from the delivery catheter the positive-locking element may disengage from the recess of the adapter piece such that the tether portion at its second end is released from the adapter piece and the loop previously formed by the tethering member is opened.

In one embodiment, the adapter piece comprises a slanted face formed on the body at a transition between the recess and the retainer groove, wherein the slanted face is formed such that the positive-locking member is guided to release the tether portion from the retainer groove in case the locking of the positive-locking member with respect to the adapter piece is released and a pulling force is exerted on the tether portion. The slanted face is formed at the transition between the recess and the retainer groove and is inclined with respect to the longitudinal direction of extension of the retainer groove. If a pulling force is exerted on the tether portion when the adapter piece is in its second position outside of the delivery catheter, hence, the positive-locking member may slide up the slanted face such that it comes out of engagement from the recess formed on the body of the adapter piece, such that the connection of the positive-locking member to the adapter piece is released.

The tethering member may be formed for example by a cable, suture or wire. The tethering member is generally flexible and hence may be flexibly bendable to form a loop for connecting the medical device to the catheter device. The tethering member may be soft, or may exhibit a substantial firmness (while allowing a sufficient bendability).

The tethering member may be fabricated from a radiopaque material such that the tethering member may be visualized for example in an X-ray examination.

An assembly comprises a catheter device of the kind described above and a medical device having a housing and a connection member arranged on the housing, wherein the tether portion of the tethering member in the connection state of the positive-locking member is connected to the connection member of the medical device. The connection member in particular may comprise an opening through which the tether portion of the tethering member extends when the medical device is connected to the catheter device.

When the positive-locking member is received on the adapter piece and is connected to the adapter piece when the adapter piece is retracted into the delivery catheter, the tethering member forms a loop, the loop providing for a connection of the medical device to the catheter device. The tether portion of the tethering member forming the loop herein interacts with the connection member of the medical device such that an operative connection in between the medical device and the catheter device is established, in particular in that the tether portion extends through an opening formed by the connection member of the medical device. Once the positive-locking member is released from the adapter piece the tether portion may be pulled out of engagement from the connection member, in particular by passing the positive-locking member through the opening formed by the connection member, such that the medical device is released from the catheter device.

The connection member of the medical device, in one embodiment, comprises a shaft bordering the opening, wherein the tether portion in the connection state extends about the shaft. The shaft may have a rounded design such that the tether portion slides on a rounded face of the shaft.

In addition, the connection member of the medical device may comprise a rounded face having, when viewed in a plane perpendicular to a longitudinal axis of the shaft, a convex curvature, the rounded face bordering the opening at a side opposite the shaft. Hence, the opening at the connection member is formed in between rounded faces, which may help to ensure that, when passing the positive-locking member through the opening for releasing the medical device from the catheter device, the positive-locking member may securely slide through the opening without being caught within the opening.

The catheter device may provide a means of navigating to and deploying a medical device in the shape of a leadless pacemaker in either the septum or the apex of the right ventricle of the heart. The catheter device herein provides a means for a user to safely and reliably steer an implantation catheter that has been preloaded with a leadless pacemaker, and navigate it across the valve from the atrium into the right ventricle, and then allow the user to safely and reliably position the catheter and implant for deployment in either the septum or atrium.

After the deployment of the implant in either location, if the user determines that the implantation site is not suitable, the user may have the means to re-sheathe the implant and reposition it to a new location and redeploy it. The catheter device leverages a tether mechanism as a way to establish a tethered connection between the medical device and the catheter device.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The various features and advantages of the present invention may be more readily understood with reference to the following detailed description and the embodiments shown in the drawings. Herein,

FIG. 1 shows a schematic view of the human heart;

FIG. 2 shows a schematic view of a leadless pacemaker device to be implanted in a patient's heart;

FIG. 3 shows a perspective view of an embodiment of a catheter device for implanting a medical device, in particular a leadless pacemaker device;

FIG. 4 shows a side view of the arrangement of FIG. 3;

FIG. 5 shows a view of a medical device having a connection member for establishing a connection to a tethering member of the catheter device;

FIGS. 6A-C show cut views of the connection member of the medical device while pulling a tether portion of the tethering member through an opening formed by the connection member for releasing the connection in between the medical device and the catheter device;

FIGS. 7A-C show perspective views of the connection member of the medical device while pulling the tether portion of the tethering member through the opening formed by the connection member;

FIG. 8 shows a view of another embodiment of a connection member of a medical device;

FIG. 9 shows a view of yet another embodiment of a connection member of a medical device;

FIG. 10 shows a view of an adapter piece of the catheter device in a position in which it is received within a delivery catheter;

FIG. 11 shows a view of an embodiment of a catheter device for implanting a medical device at an implantation site within a patient;

FIG. 12 shows a view of articulation of zones of a steerable catheter of the catheter device;

FIG. 13 shows an enlarged view of a distal portion of the catheter device;

FIG. 14A shows a schematic cross-sectional view through the steerable catheter of the catheter device;

FIG. 14B shows a schematic longitudinal view of the steerable catheter;

FIG. 15A shows a schematic cross-sectional view through the steerable catheter of the catheter device, according to another embodiment;

FIG. 15B shows a schematic longitudinal view of the steerable catheter of FIG. 15A;

FIG. 16 shows an enlarged view of a proximal portion of the catheter device including a handle device;

FIG. 17 shows the catheter device when introduced through an introducer during implantation;

FIGS. 18A-D show the catheter device when navigated across a tricuspid valve to enter the right ventricle of a patient's heart;

FIG. 19 shows the catheter device when further introduced through an introducer during implantation;

FIGS. 20A-C show the catheter device when navigated across a tricuspid valve to enter the right ventricle of a patient's heart;

FIG. 21 shows the catheter device during deployment of the medical device at an implantation site; and

FIGS. 22A-E show different views while implanting a medical device using a catheter device.

### DETAILED DESCRIPTION

Subsequently, embodiments of the present invention shall be described in detail with reference to the drawings. In the drawings, like reference numerals designate like structural elements.

It is to be noted that the embodiments are not limiting for the present invention, but merely represent illustrative examples.

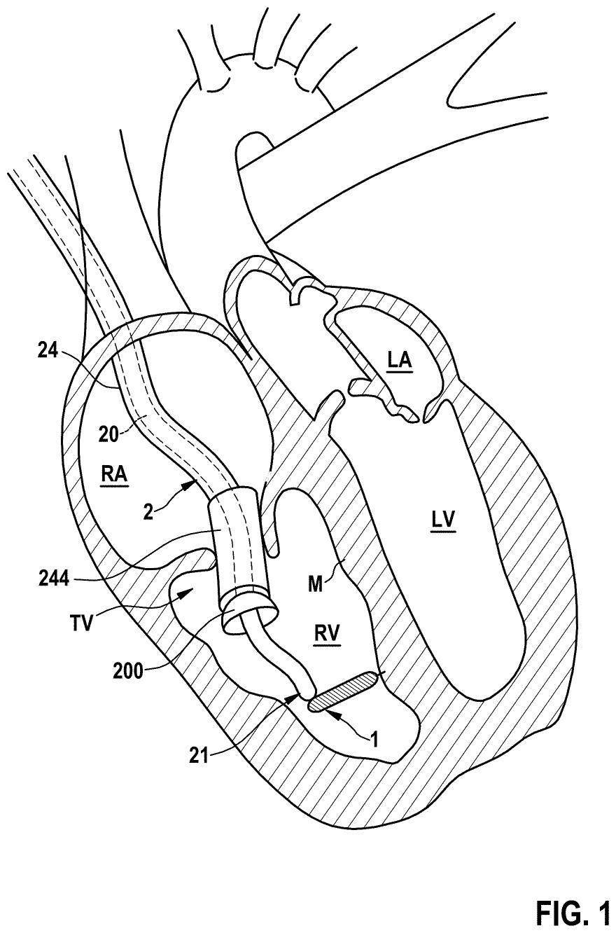

FIG. 1 shows, in a schematic drawing, a human heart comprising a right atrium RA, a right ventricle RV, a left atrium LA and a left ventricle LV, an implantable medical device 1 being implanted into the right ventricle RV, the implantable medical device 1 for example having the shape of a leadless pacemaker device for providing a pacing of the heart's activity at the right ventricle RV.

For implantation the implantable medical device 1 by means of a catheter device 2 is placed at an implant location, for example in the right ventricle RV—as illustrated in FIG. 1—such that it comes to rest on myocardial tissue M, for example at the apex of the right ventricle RV or at the septum.

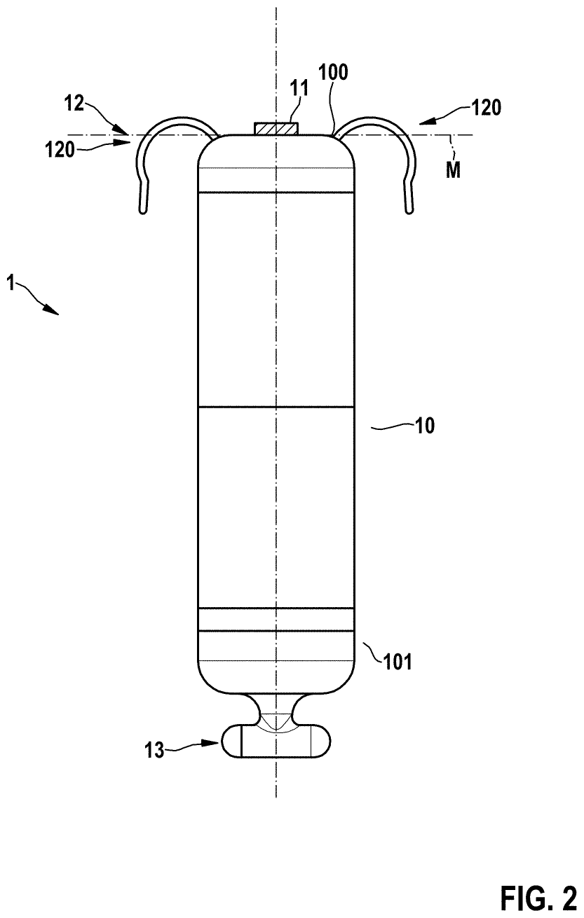

FIG. 2 shows, in a schematic drawing, an example of an implantable medical device 1 in the shape of a leadless pacemaker device, the implantable medical device 1 having a housing 10 forming a proximal end 101 and a distal end 100, the implantable medical device 1 being configured to be placed on myocardial tissue M by means of its distal end 100. On the distal end 100, herein, an electrode device 11 is placed which, potentially together with other electrodes of the implantable medical device 1, serves to provide for a pacing action in order to stimulate cardiac activity in a defined manner.

During implantation the implantable medical device 1 is to be placed at an implant location, for example on myocardial tissue M, and is to be fastened to tissue at the implant location. For this, the implantable medical device 1 comprises an anchoring device 12 having a multiplicity of anchoring members 120 in the shape of tines, wires or tubes or the like, the anchoring members 120 extending from the housing 10 of the implantable medical device 1 at the distal end 100 in order to engage with tissue for fastening the implantable medical device 1 to the tissue.

For implantation the implantable medical device 1 is to be delivered towards the implant location by means of a catheter device 2, the catheter device 2 comprising a delivery catheter 20 guided within a steerable catheter 24. The catheter device 2 for implantation is guided for example through the inferior vena cava into the patient's heart to access, via the right atrium RA and across the tricuspid valve TV, the right ventricle RV. During delivery the implantable medical device 1 is fixed to the delivery catheter 20, but is to be released once the implantable medical device 1 has reached the implant location and is fixed to tissue in the region of the implant location.

When the implantable medical device 1, by means of its distal end 100, is placed on tissue at the implant location, the anchoring members 120 of the anchoring device 12 come into engagement with the tissue such that the medical device 1 is mechanically fixed to the tissue. Herein, once the medical device 1 is placed on the tissue at the implant location, a testing is to be performed in order to ensure the mechanical fixation of the medical device 1 to the tissue and in addition to test the proper functionality of the medical device 1, in particular for performing a pacing operation for example in the right ventricle RV of the patient's heart.

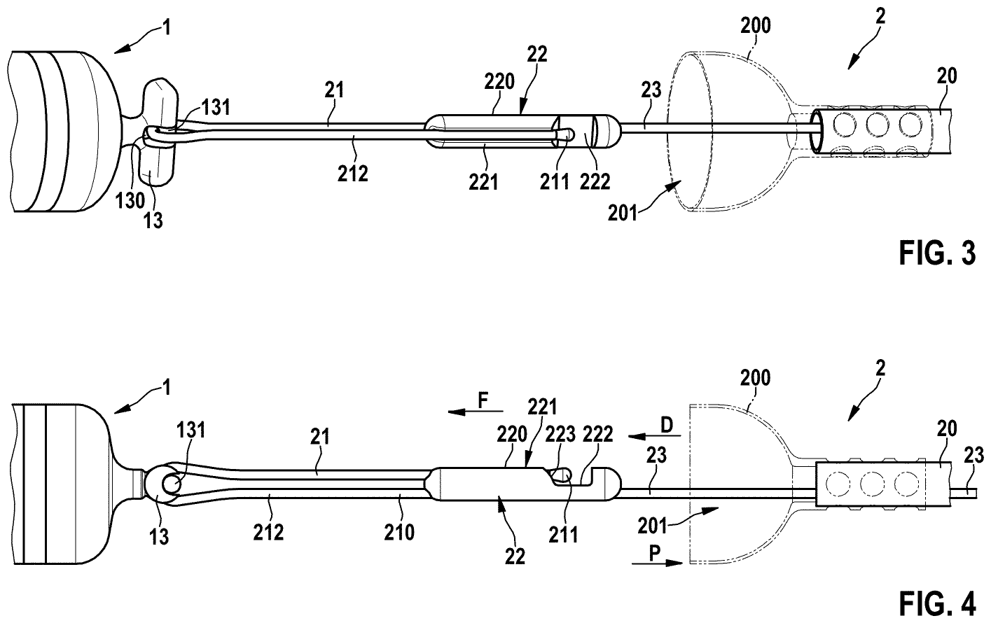

Referring now to FIGS. 3 and 4, an embodiment of a catheter device 2 comprises a delivery catheter 20 generally extending along a longitudinal direction and being received in a steerable catheter 24, as shall be explained further below.

The delivery catheter 20 comprises an inner lumen 201 in which, in the illustrated embodiment, a mandrel 23 is received, the mandrel 23 extending through the entire length of the delivery catheter 20 towards a proximal end of the catheter device 2. The mandrel 23 is sufficiently flexible such that it may be deformed together with the delivery catheter 20 when guiding the catheter device 2 through body portions of the patient, but at the same time is flexurally rigid and kink-proof such that it may be axially moved with respect to the delivery catheter 20.

The catheter device 2 furthermore comprises an adapter piece 22 fixedly connected to the mandrel 23, for example by welding. The adapter piece 22 has a generally longitudinal shape with rounded tips and is designed such that it may be received within the inner lumen 201 of the delivery catheter 20 when it is retracted into the delivery catheter 20 by means of the mandrel 23.

The adapter piece 22 is part of a tethering mechanism comprising a tethering member 21, which at an end 210 is fixedly connected to the adapter piece 22, for example by welding. The tethering member 21 has a tether portion 212 extending from the adapter piece 22 and, at an end opposite to the end 210, is adjoined by a positive-locking member 211 in the shape of a spherical ball, as visible from FIGS. 3 and 4.

In a connection state, the positive-locking member 211 is received within a recess 222 formed on a body 220 of the adapter piece 22. In the connection state, herein, the tether portion 212 is received within a retainer groove 221 adjoining the recess 222, the retainer groove 221 having a width such that the tether portion 212 may be snuggly placed within the retainer groove 221, but the positive-locking member 211 having a wider diameter is prevented from passing through the retainer groove 221.

The tethering member 21 serves to establish a connection in between a medical device 1 to be implanted and the catheter device 2. In the connection state the positive-locking member 211 at the far end of the tether portion 212 of the tethering member 21 is received in the recess 222 of the adapter piece 22, and by retracting the adapter piece 22 into the inner lumen 201 of the delivery catheter 20 the form locking connection in between the adapter piece 22 and the positive-locking member 211 is locked.

This is illustrated in FIG. 10. In a position in which the adapter piece 22 is received within the inner lumen 201 of the delivery catheter 20 the circumferential wall of the delivery catheter 20 surrounds the adapter piece 22, such that a transverse movement of the positive-locking member 211 to disengage from the recess 222 of the adapter piece 22 is prevented and the connection in between the positive-locking member 211 and the adapter piece 22 is thus locked.

In this respect it should be noted that circumferential wall of the delivery catheter 20 should have a structural stiffness such that the ball-shaped locking member 211 cannot deform the wall towards the outside to disengage from the adapter piece 22. In particular, the wall shall comprise such a stiffness that the tether portion 212 cannot disengage from and be pulled off the adapter piece 22.

When the positive-locking member 211 is received on the adapter piece 22 and when the adapter piece 22 is retracted into the inner lumen 201 of the delivery catheter 20, the tether portion 212 of the tethering member 21 forms a loop which extends through an opening 130 formed on a connection member 13 of the implantable medical device 1, as this is illustrated in FIGS. 3 and 4. By means of the tethering member 21, hence, a connection in between the catheter device 2 and the medical device 1 is established, such that the medical device 1 by means of the catheter device 2 may be advanced towards an implant location and may be placed on tissue in the region of the implant location.

In a delivery state, herein, the adapter piece 22 is retracted into the delivery catheter 20 in a proximal direction P as indicated in FIG. 4 such that the medical device 1 is drawn towards an alignment device 200 fixedly connected to the delivery catheter 20, such that the medical device 1 is received on the alignment device 200. Hence, a firm, aligned connection in between the medical device 1 and the delivery catheter 20 is established. In this delivery state the medical device 1 by means of the catheter device 2 may be advanced towards an implant location of interest.

Once the medical device 1 has reached the implant location and, by means of the anchoring device 12, has engaged with tissue at the implant location, the medical device 1 shall be released from the catheter device 2. The release herein shall take place in phases in order to, in an initial release phase, allow for a testing on the medical device 1 and, in a second release phase, then fully release the medical device 1 from the catheter device 2 such that the catheter device 2 may be removed while the medical device 1 remains at the implant location.

In the initial release phase, the adapter piece 22 is moved, within the inner lumen 201 of the delivery catheter 20, in a distal direction D as illustrated in FIG. 4, such that the tether portion 212 exits from the delivery catheter 20 while the adapter piece 22 still remains within the inner lumen 201 of the delivery catheter 20. The medical device 1 hence is spatially removed from the alignment device 200 of the delivery catheter 20, while the connection via the tethering member 21 still remains in effect.

In this initial release phase a fixation assessment (a so-called tug test) may be performed by pulling on the medical device 1 in order to ensure a proper fixation of the medical device 1 on tissue by means of the anchoring device 12. Such tug test may for example be performed by moving the mandrel together with the adapter piece 22 in the proximal direction P. In addition, an electrical testing of the functionality of the medical device 1, for example a leadless pacemaker device, may be performed to ensure a proper functionality of the medical device 1, while the medical device 1 is allowed to sit freely on tissue and substantially is not affected by a force action of the tethering member 21.

If during this testing it is found that the medical device 1 does not securely hold on the tissue, but is released when applying a defined force during the tug test, or if it is found that the medical device 1 is not positioned correctly for performing its electrical functioning, the medical device 1 may have to be repositioned. For this, the adapter piece 22 may again be retracted into the delivery catheter 20 by pulling the mandrel 23 and together with the mandrel 23 the adapter piece 22 in the proximal direction P such that the medical device 1 again comes to engage with the alignment device 200. The medical device 1 may then be positioned at another location, and testing may be repeated until a proper positioning and functioning of the medical device 1 is confirmed.

Once it is found that the medical device 1 is properly implanted and fixed on tissue and in addition functions correctly, the medical device 1 is to be released from the tethering member 21. For this, the adapter piece 22 is, by pushing on the mandrel 23 in the distal direction D, moved out of the inner lumen 201 of the delivery catheter 20, as this is shown in FIGS. 3 and 4. In such position of the adapter piece 22 the positive-locking member 211 on the far end of the tether portion 212 no longer is blocked within the recess 222 formed on the body 220 of the adapter piece 22, such that the positive-locking member 211 may be released from the adapter piece 22 and the loop formed by the tethering member 22 may hence be opened.

The releasing of the positive-locking member 211 from the adapter piece 22 herein is supported by a slanted face 223 at the transition of the recess 222 and the retainer groove 221, as this is apparent from FIG. 4. When exerting a pulling force F on the tether portion 212 the positive-locking member 211 may slide up the slanted face 223 and may hence disengage from the recess 222, such that also the tether portion 212 comes out of engagement from the retainer groove 221. The tether portion 212 at its far end hence is released from the adapter piece 22.

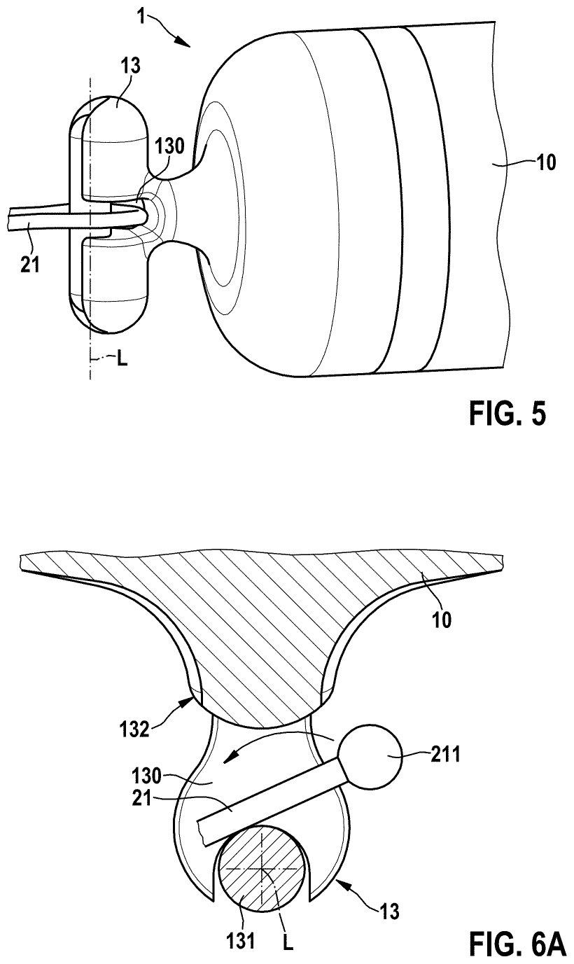

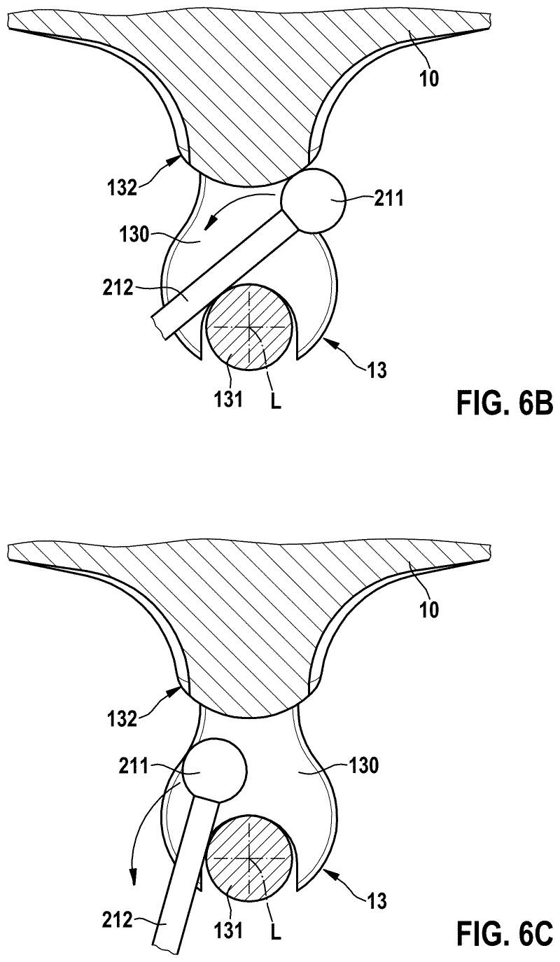

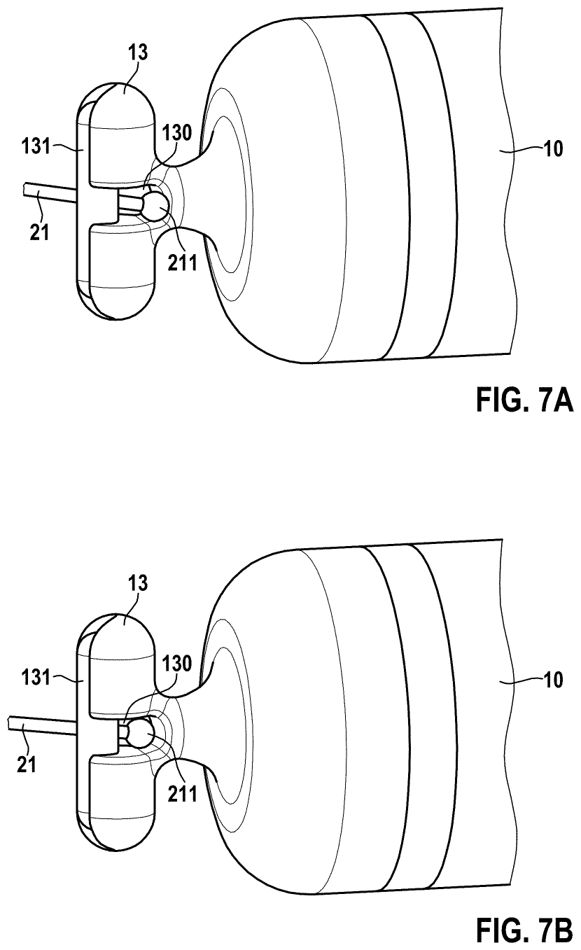

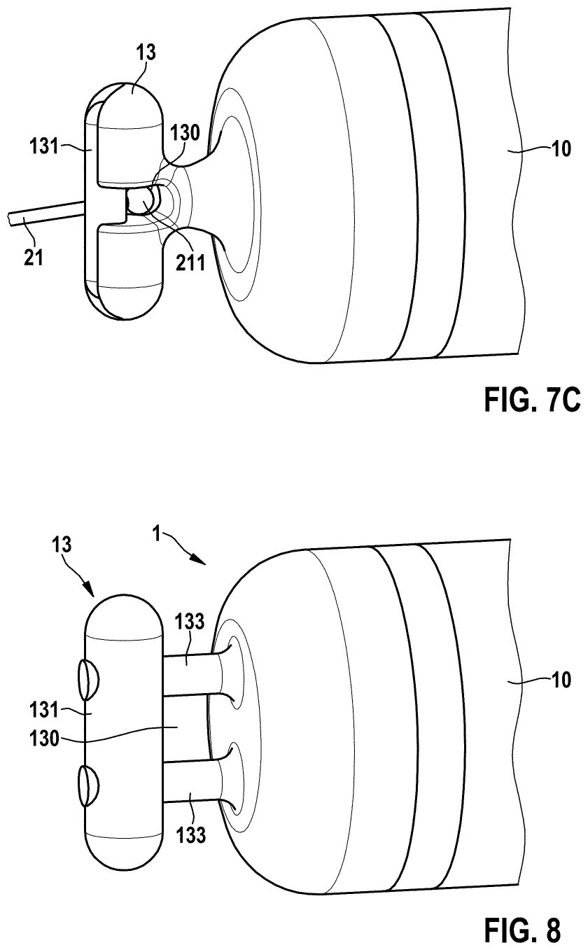

In the connection state, the tether portion 212 extends through an opening 130 of the connection member 13 of the medical device 1, as this is illustrated in FIG. 5. When the positive-locking member 211 is to be released from the adapter piece 22, the tethering member 21 may be pulled through the opening 130 to disconnect from the connection member 13, as illustrated in FIGS. 6A to 6C and 7A to 7C. In particular, when pulling on the tethering member 21 the positive-locking member 211 is passed through the opening 130 of the connection member 13 and is moved around a shaft 131 of the connection member 13 about which the tether portion 212 extends in the connection state.

Once the positive-locking member 211 is passed through the opening 130, the medical device 1 is free of the tethering member 21 and the connection in between the catheter device 2 and the medical device 1 is fully released.

As visible from FIGS. 6A to 6C and 7A to 7C, the shaft 131 extending along a longitudinal axis L is rounded. In addition, the opening 130 at a side opposite to the shaft 131 is bordered by a rounded face 132 having a convex curvature, when viewed along a plane perpendicular to the longitudinal axis L. Because the opening 130, hence, is bordered, in the plane perpendicular to the longitudinal axis L of the shaft 131, by rounded faces, the positive-locking member 211 may safely pass through the opening 130, at a minimum risk of being caught within the opening 130.

For pulling the tethering member 21 through the opening 130, the mandrel 23 together with the adapter piece 22 is moved in the proximal direction P into the delivery catheter 20. Once the connection to the medical device 1 is fully released, the delivery catheter 20 may be removed from the patient.

The tethering member 21 may be made of a radiopaque material such that the positioning of the tethering member 21 may be visualized by an X-ray technology during implant.

In the embodiment of the connection member 13 of the medical device 1 of FIG. 5 and FIGS. 6A to 6C, 7A to 7C, the shaft 131 is for example received within a slot at a distal face of the connection member 13 and is welded to the connection member 13 within the slot. In the connected state the tether portion 212 of the tethering member 221 extends about the shaft 131 such that the medical device 1 is fixedly connected to the catheter device 2.

In another embodiment shown in FIG. 8 a shaft 131 is connected to a housing 10 of the medical device 1 by means of two posts 133, the opening 130 being formed in between the posts 133 and being bordered by the shaft 131. The posts 133, in the embodiment of FIG. 8, engage with mounting openings of the shaft 131 and are for example welded to the shaft 131.

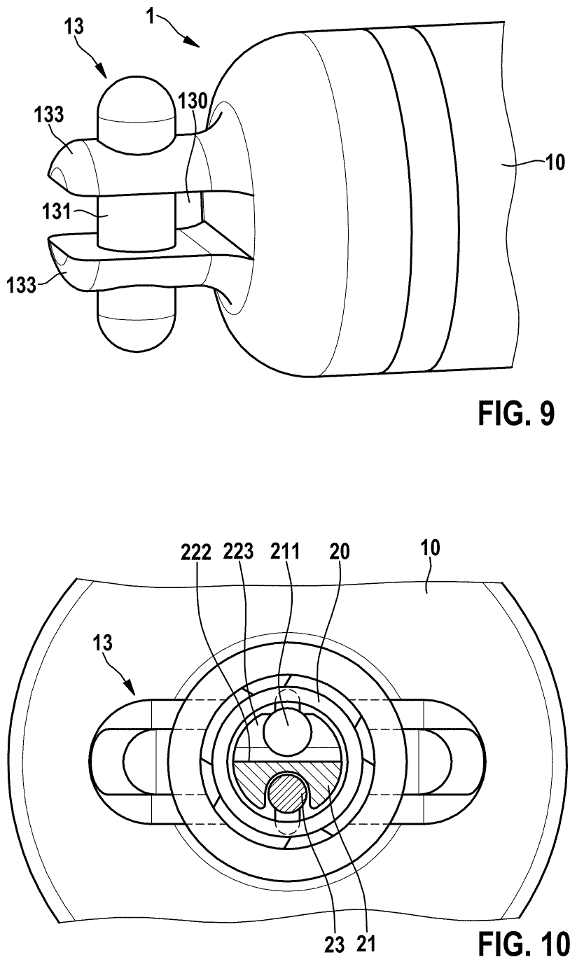

In a different embodiment shown in FIG. 9, a shaft 131 is received within mounting openings of two posts 133, projecting distally from the housing 10 of the medical device 1, the opening 130 again being formed in between the posts 133 and being bordered by the shaft 131.

Referring now to FIG. 10, in a position in which the adapter piece 21 is retracted into the delivery catheter 20 the adapter piece 21 is snugly received within the inner lumen 201 of the delivery catheter 20. The positive-locking member 211 received within the recess 222 formed on the body 220 of the adapter piece 22 hence is blocked and prevented to disengage from the recess 222, such that a positive-locking connection in between the adapter piece 22 and the positive-locking member 211 on the tether portion 212 is established. By moving the adapter piece 22 in the distal direction D out of the inner lumen 201 of the delivery catheter 20, the blocking of the positive-locking member 211 within the recess 222 is released, such that the positive-locking member 211 can disengage from the recess 222 for disconnecting the catheter device 2 from the medical device 1.

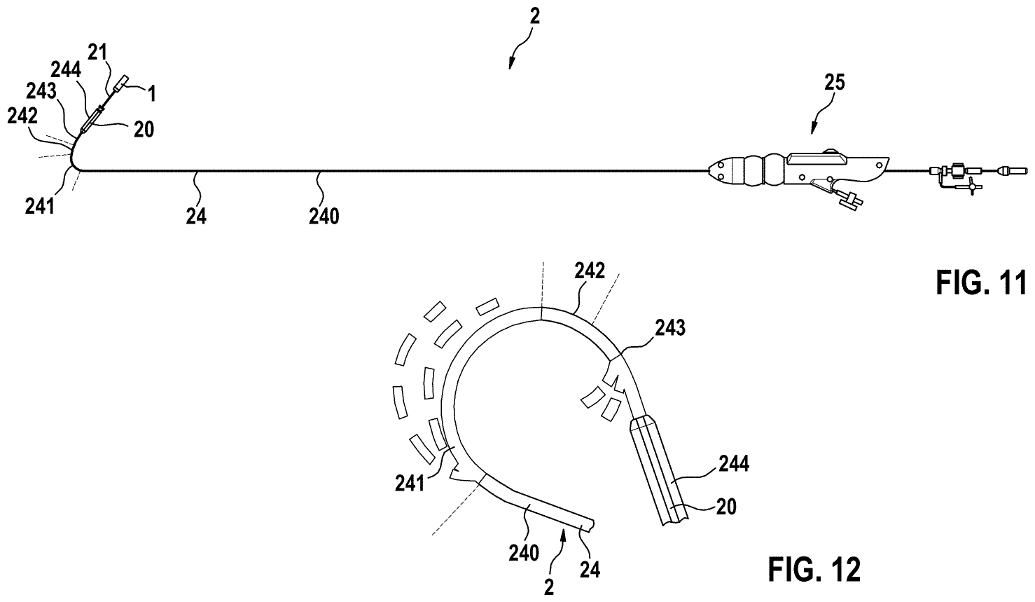

Referring now to FIG. 11, the catheter device 2 comprises a steerable catheter 24 which receives and guides the delivery catheter 20 within. The steerable catheter 24 herein provides for a steering function in order to navigate the catheter device 2 into the patient and towards an implantation site in order to deliver and deploy the medical device 1 towards the implantation site.

As shown in FIG. 12 in view of FIG. 11, the steerable catheter 24 has the shape of a longitudinal tube which comprises a straight shaft section 240 adjoined by a first steering articulation zone 241, which is adjoined by a short, straight connection section 242, which is adjoined by a second steering articulation zone 243 at a distal end of the steerable catheter 24. The second steering articulation zone 243 herein is connected to a protective cup 244, which serves to receive and sheathe the medical device 1 during implantation such that, when the catheter device 2 is advanced into the patient for implanting the medical device 1, the medical device 1 is housed and enclosed within the protective cup 244.

A steering by means of the steerable catheter 24 is achieved using the steering articulation zones 241, 243. Herein, as shown in an enlarged view of a proximal portion of the catheter device 2, the first steering articulation zone 241 is arranged proximally with respect to the second steering articulation zone 243 and comprises a length L1 which is larger than a length L2 of the second steering articulation zone 243. In the respective steering articulation zone 241, 243 the steerable catheter 24 may deflect such that the catheter device 2 may be navigated through tighter structures within the patient, for example across the tricuspid valve TV in order to enter the right ventricle RV of the patient's heart to deliver a medical device 1 towards an implantation site for example at the apex or the septum of the right ventricle RV.

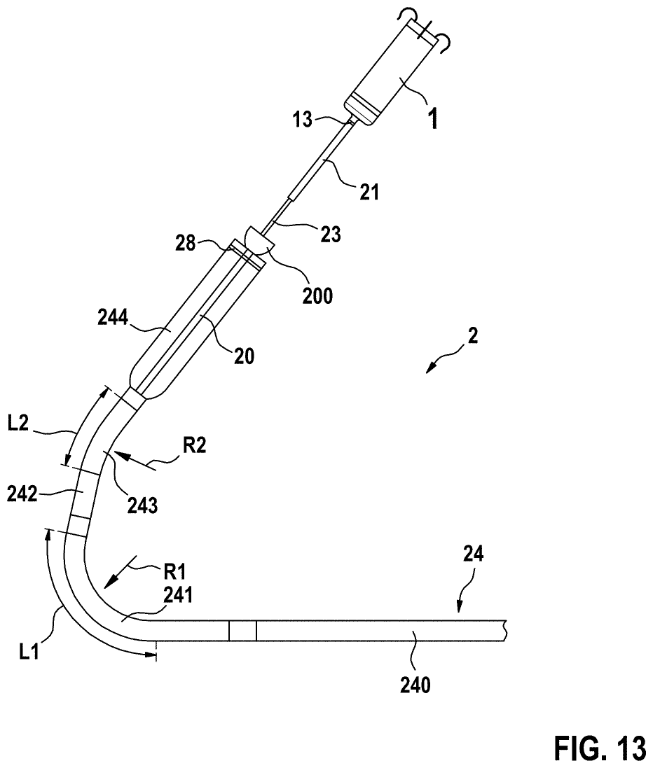

As visible from FIG. 13, the steering articulation zones 241, 243 are configured to provide for a deflection having different bending radii R1, R2. In particular, due to the larger length L1 of the first steering articulation zone 241 a bending at the first steering articulation zone 241 takes place substantially at a first bending radius R1, whereas a bending at the second steering articulation zone 243 takes place substantially at a second bending radius R2 smaller than the first bending radius R1. Hence, at the second steering articulation zone 243 a tight bending with a small bending radius R2 may be achieved, whereas in the first steering articulation zone 241 a bending at a substantially larger bending radius R1 may be achieved.

The steering articulation zones 241, 243 may be actively or passively deflectable for providing for a steering.

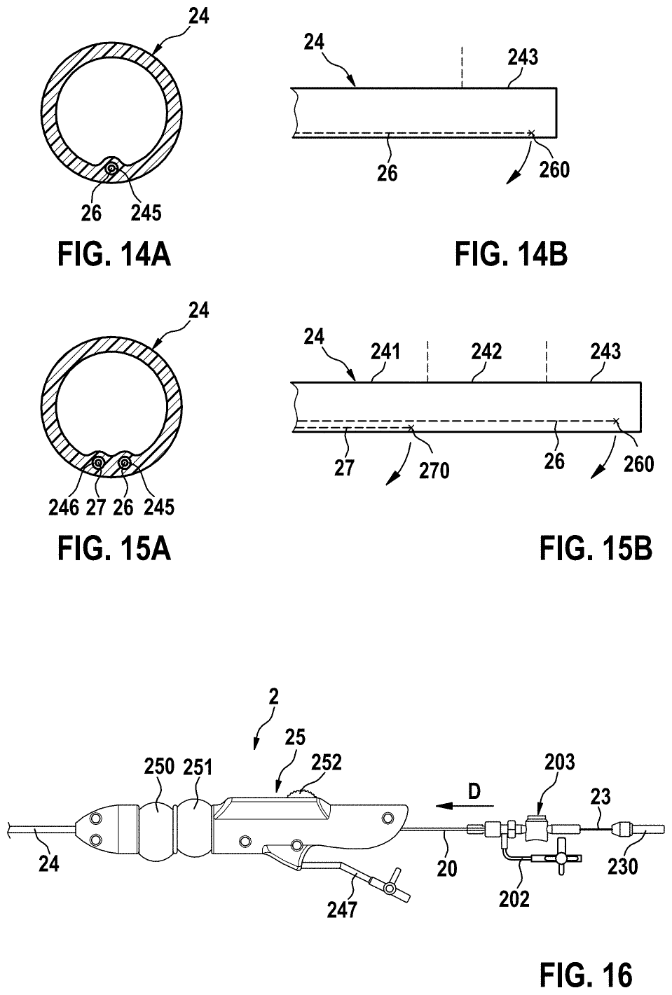

An active steering may for example be achieved using steering devices 26, 27 in the shape of pull wires, as shown in FIGS. 14A, 14B, 15A, and 15B. The steering devices 26, 27 are associated with the steering articulation zones 241, 243 and extend along the steerable catheter 24 towards a proximal handle device 25.

In one embodiment, as shown in FIGS. 14A and 14B, only one of the steering articulation zones 241, 243 is actively steerable using an associated steering device 26, 27, for example the second steering articulation zone 243. In this case, the other steering articulation zone, for example the proximal steering articulation zone 241, may be passively steerable by forming the steering articulation zone as a shape-set curve having a predefined, preshaped curvature.

The steering device 26 associated with the actively steerable steering articulation zone 243 in this case at a fixing location 260 is connected to the steerable catheter 24, as shown in FIG. 14B, and is guided within an inner lumen 245 e.g. in the wall of the steerable catheter 24 towards the proximal end of the catheter device 2, as shown in FIG. 14A, such that a pulling action on the steering device 26 may cause a deflection at the steering articulation zone 243.

In another embodiment, as shown in FIGS. 15A and 15B, each steering articulation zone 241, 243 is associated with a steering device 26, 27, each steering device 26, 27 being connected within the associated steering articulation zone 241, 243 to the steerable catheter 24 at a fixing location 260, 270, as shown in FIG. 15B, and being guided within a guide lumen 245, 246 formed for example in the wall of the steerable catheter 24, as shown in FIG. 15A.

The steering articulation zones 241, 243 may be actively deflectable in one direction using a single steering device 26, 27. Alternatively, one or both of the steering articulation zones 241, 243 may be deflectable in two or more directions, such that the steerable catheter 24 in the respective steering articulation zones 241, 243 may deflect in a plane perpendicular to the longitudinal axis of extension of the catheter device 2.

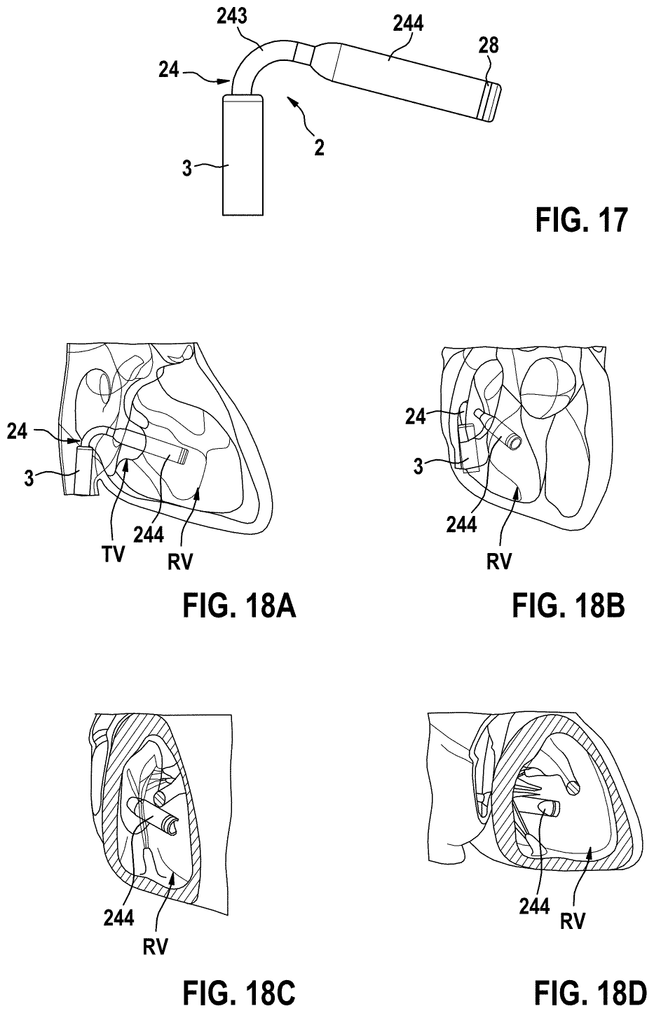

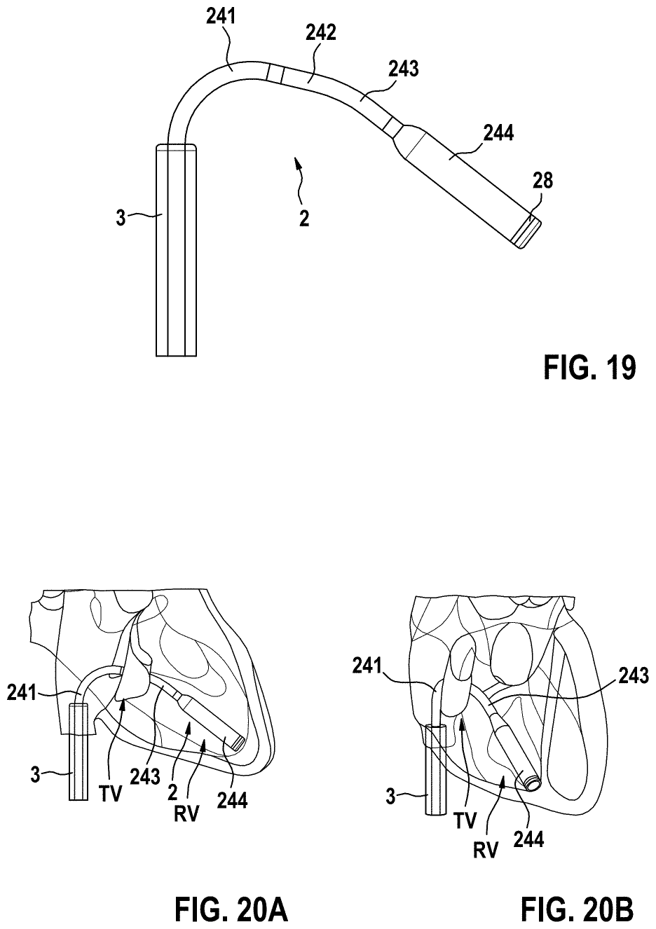

In another embodiment, one or both of the steering articulation zones 241, 243 may be passively deflectable in that one or both of the steering articulation zones 241, 243 are formed by shape-set curves. In this case, the respective steering articulation zone 241, 243 has a predefined, preshaped curvature such that the respective steering articulation zone 241, 243 in a relaxed state without forces acting onto the steering articulation zone 241, 243 assumes its predefined bent shape. In this case, a deflection for steering and navigating the catheter device 2 may be caused in combination with an introducer 3 (see for example FIGS. 17 to 20A-20C), wherein the respective steering articulation zone 241, 243 may for example be straightened when introduced through the introducer 3 (which has a straight, cylindrical hollow shape), but reverts to its predefined deflection when exiting the introducer 3 at a distal end, as shown in FIGS. 17 and 19. This may be used to navigate the catheter device 2 through tight structures, such as the tricuspid valve TV for entering the right ventricle RV of the patient's heart.

FIG. 16 shows an embodiment of a handle device 25 of the catheter device 2 comprising actuation members 250-252 for controlling operation of the catheter device 2.

In the shown embodiment, the handle device 25, by means of the actuation members 250, 251, serves to actively control a deflection at the two steering zones 241, 243 in that the steering articulation zones 241, 243 may be actively deflected by adjusting the actuation members 250, 251. In the shown embodiment the actuation members 250, 251 have the shape of steering knobs which may be rotated on the handle device 25 in order to act onto steering devices in the shape of pull wires 26, 27 in order to exert a pulling action on the steering articulation zones 241, 243.

An additional actuation member 252 serves to adjust the delivery catheter 20 axially with respect to the steerable catheter 24 in particular for deploying the medical device 1 upon reaching an implantation site. The actuation member 252 has the shape of a slider which may be axially moved on the handle 25 in order to advance the delivery catheter 20 proximally for deploying the medical device 1.

The delivery catheter 20 extends through the handle device 25 and through the steerable catheter 24. At its proximal end the delivery catheter 20 herein is connected to a tether control 203 for controlling movement of the mandrel 23 with respect to the delivery catheter 20. The tether control 203 may in particular be configured to prevent an unintentional actuation of the mandrel 23, the tether control 203 being configured to prevent, in a delivery state of the catheter device 2, that the mandrel 23 may be pushed into the delivery catheter 20 in the distal direction D, by which movement otherwise the adapter piece 22 may be caused to exit from the delivery catheter 20 allowing the positive-locking element 211 hence to disengage from the adapter piece 22.

The tether control 203 in combination with the mandrel 23 and a wire clamp 230 arranged on the mandrel 23 may be designed such that the mandrel 23, in a non-actuated state of the tether control 203, can be moved in the distal direction D with respect to the delivery catheter 20 only such far that the adapter piece 22 at the distal end of the delivery catheter 20 cannot exit from the delivery catheter 20. By means of the tether control 203 hence a release of the positive-locking member 211 from the adapter piece 22 is prevented.

By actuating the tether control 203 the mandrel 23 may be released such that the mandrel 23 may be passed through the tether control 203 and may be advanced further in the distal direction D through the delivery catheter 20 to cause the adapter piece 22 to exit from the delivery catheter 20 for releasing the medical device 1 from the catheter device 2.

For implantation the catheter device 2 may be advanced into a patient using an introducer 3, as this is shown in FIG. 17 and FIGS. 18A to 18D. The introducer 3 has a cylindrical shape and forms a hollow, straight channel through which the catheter device 2 may be passed for implanting the medical device 1.

The introducer 3 in the example of FIGS. 18A to 18D is introduced at an incision site towards the right atrium of the patient's heart. By passing the catheter device 2 through the introducer 3 the catheter device 2—with the medical device 1 received within the protective cup 244 at the distal end of the catheter device 2—is advanced into the patient's heart. For this, once the second, distal steering articulation zone 243 exits the distal end of the introducer 3 it is deflected such that the protective cup 244 with the medical device 1 received therein is navigated through the tricuspid valve TV into the right ventricle RV.

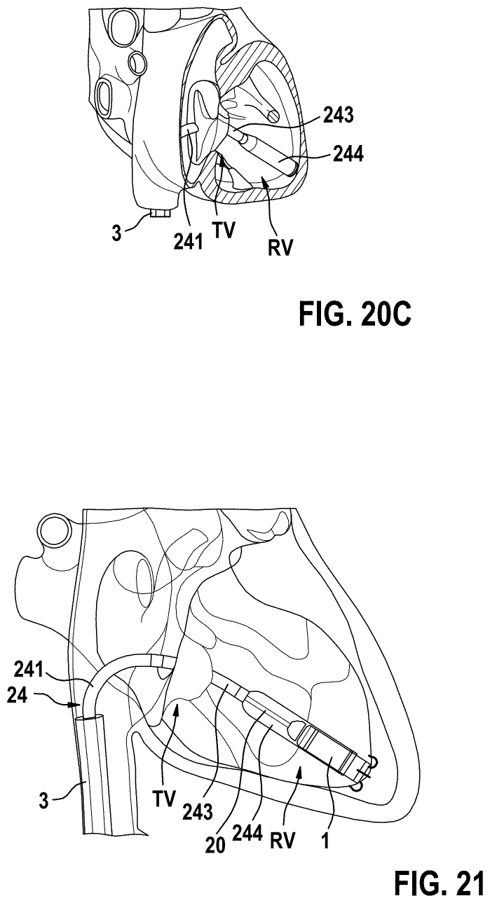

When further advancing the catheter device 2, a further steering may be achieved once the first, proximal steering articulation zone 241 exits from the introducer 3, as shown in FIG. 19 and FIGS. 20A to 20C, such that the protective cup 244 with the medical device 1 received therein may be advanced towards an implantation site, for example in the region of the apex of the right ventricle RV.

As visible from FIGS. 13, 17 and 19, a radiopaque marker 28 such as a radiopaque ring is arranged on a distal end of the protective cup 244, such that the advancing of the catheter device 2 may take place under the control of medical imaging using an X-ray technology.

Once the implantation site is reached, the medical device 1 is deployed, as shown in FIG. 21. For this, the delivery catheter 20 is advanced distally within the steerable catheter 24, which at the same time may be retracted to some extent, such that the medical device 1 is pushed out of the protective cup 244 and the anchoring members 120 arranged distally on the medical device 1 come into engagement with tissue at the implantation site for anchoring the medical device 1 to the tissue.

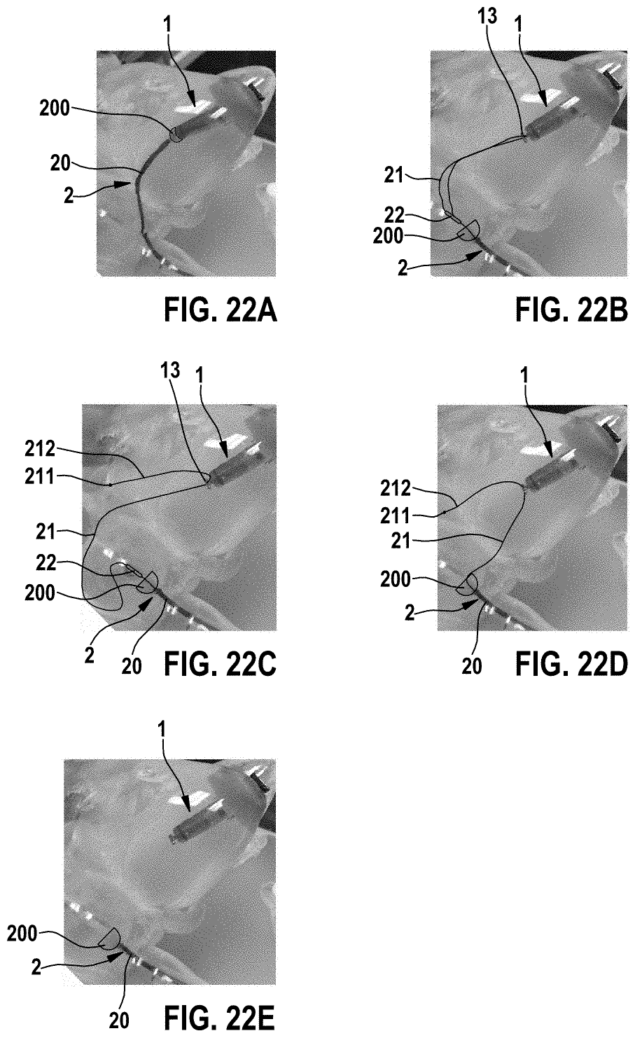

FIGS. 22A to 22 B illustrate a release procedure for releasing the medical device 1 on tissue at an implantation site.

During delivery, as illustrated in FIG. 22A, the medical device 1 is received on the alignment device 200 of the delivery catheter 20 and, by means of the tethering mechanism comprised of the tethering member 21, the adapter piece 22 and the mandrel 23, is fixedly connected to the delivery catheter 20. In this delivery state the medical device 1 may be advanced towards an implant location and may be placed on tissue in the region of the implant location.

Once the implant location is reached and the medical device 1 is deployed from the protective cup 244 and hence is placed on tissue in the region of the implant location, as illustrated in FIG. 22B, the adapter piece 22 is moved distally in the delivery catheter 20 such that the tethering member 21 exits from the delivery catheter 20 while the adapter piece 22 still remains within the delivery catheter 20. The medical device 1 hence is spatially removed from the alignment device 200 of the delivery catheter 20. In this state the medical device 1 is allowed to sit freely on tissue in the region of the implant location, allowing a user to perform a fixation assessment (a so-called tug test) and an electrical testing of the medical device 1.

Once the testing has successfully been concluded, the adapter piece 22 is further advanced in the distal direction D by moving the mandrel 23 until the adapter piece 22 exits from the delivery catheter 20, as this is illustrated in FIG. 22C. In this state the positive-locking member 211 at the far end of the tether portion 212 of the tethering member 21 becomes free of the adapter piece 22.

By now retracting the adapter piece 22 in the proximal direction P into the delivery catheter 20, as illustrated in FIG. 22D, the tethering member 21 is pulled in the proximal direction P such that the positive-locking member 211 is pulled through the opening 130 of the connection member 13 of the medical device 1.

In a release state, shown in FIG. 22E, the medical device 1 is free of the catheter device 2, such that the catheter device 2 may be removed while the medical device 1 remains in place at the implant location.

If during the testing it is found that the medical device 1 is not properly placed on tissue and/or that the implantation site potentially is not suitable for the medical device 1, the medical device 1 may again be pulled tight towards the alignment device 200 and may be re-sheathed by advancing the protective cup 244 over the medical device 1, such that the medical device 1 may be repositioned and placed at a different implantation site or may be removed entirely from the patient anatomy.

In one embodiment, the catheter device generally comprises a delivery catheter that is connected to the implant protective cup that is used to sheathe and unsheathe the implant and its fixation mechanism. The delivery catheter has two distinct articulation areas.

The distal steering articulation zone is a short length tight radius steering deflection along the xy axis that is used primarily for crossing the tricuspid valve efficiently and for deploying the implant into the mid to lower septum. The embodiments for the distal articulation zone could comprise either an articulatable distal curve in a single direction via the handle (current design embodiment), an articulatable bi-directional distal curve via a handle actuator, or a shape-set distal curve with no articulation on the handle.

The proximal steering articulation zone, in one embodiment, is a longer length relaxed radius steering deflection along the xy axis that is used primarily for navigating to and deploying the implant into the apex within the right ventricle. The embodiments for this proximal articulation zone could comprise either an articulatable proximal curve via a handle actuator, or a shape-set proximal curve with no articulation via a handle actuator.

The catheter design is also designed to incorporate a tether mechanism technology.

The handle design includes a means to steer each steerable articulation zone independently from one another. One embodiment comprises two steering knobs on the handle. However, another means would be a single steering knob or actuator that could toggle between each steering deflector. The handle is mechanically designed to that when the user removes their hand from the handle, the deflection of the catheter at any given point is maintained and no relaxation of the catheter to its zero state occurs.

Lastly, the handle and catheter is designed with a slider to deploy the implant by pushing the implant out of the implant protective cup and into the heart wall. The implant is designed to be attached to the catheter via the tether mechanism which pulls the implant flush against the alignment cup/non-steerable delivery catheter. The non-steerable delivery catheter is mechanically attached to the deployment slider. When the deployment slider is advanced distally from its proximal starting/sheathed position, the non-steerable delivery catheter is advanced distally at the same rate which pushes and deploys the implant out of the implant protective cup.

The present design may comprise one or more (or all) of the following features:
A delivery catheter that has a high degree of control and maneuverability via the incorporation of two independent xy-axis steering articulation zones that are run in series that allows the user the ability to perform leadless pacemaker implantation into both the septum and the apex within the right ventricle while also allowing the user the ability to cross the tricuspid valve safely and reliably.
   a) The embodiments for the distal articulation zone could comprise either an articulatable distal curve in a single direction via the handle (current design embodiment), an articulatable bi-directional distal curve via a handle actuator, or a shape-set distal curve with no articulation on the handle.
   b) The embodiments for this proximal articulation zone could comprise either an articulatable proximal curve via a handle actuator (current design embodiment), or a shape-set proximal curve with no articulation via a handle actuator.
An inner non-steerable delivery catheter that is used to push the implant out of the implant protective cup for deployment and retract the implant back into the implant protective cup (re-sheathing) for repositioning and redeployment.
A tether mechanism technology may be implemented.
The means for the delivery catheter articulation zones to maintain their articulated positions when the user's hands are no longer touching the catheter handle.
The ability for the user to re-sheathe the implant into the outer catheter's implant protective cup safely and reliably.

The ability for the user to re-sheathe, reposition and redeploy the implant within a newly chosen implantation site in either the septum or apex.
The ability for the user to perform a mechanical tug test of the implant to assess tine fixation within the new implantation site prior to release of the implant and disengagement of the tether between the implant and catheter.

The concept associated with the present disclosure may provide a dedicated tool designed to safely and reliably navigate to and deploy a leadless pacemaker into either the septum or apex in the right ventricle. The concept associated with the present disclosure accomplishes this by utilizing two different articulation zones to steer the catheter in the xy-axis independently for safe and reliable crossing of the tricuspid valve and then navigation to either the mid/lower septum or the apex. The different dual zones may offer a user increased steering control and the ability to safely and reliably access the septum for deployment without manipulation or goose-necking of the catheter which may introduce excessive patient risks or harm. The distal articulation zone is designed to have a tight radius which can overcome difficult geometries present, especially in smaller Asian anatomies, which can hinder the ability for a single articulation curve to cross the tricuspid valve. Furthermore, it allows the user the ability to access the septum using this curve alone. If the user wishes to not deploy the implant into the septum, they can then utilize the proximal steering articulation curve to navigate to the apex for deployment.

The present disclosure may also incorporate and utilize a tether mechanism to maintain a connection to the implant until the user is sure that the implantation site location is acceptable, at which point they can safely release the tether and remove the catheter tooling.

If the user decides that the implantation site location is not acceptable, the present disclosure allows the user to safely and reliably re-sheathe the implant and reposition and redeploy it into a new location.

Another advantage of this present disclosure may include means to push the implant into the heart wall for maximum fixation mechanism engagement efficiency rather than rest the implant against the heart wall and retract the protective cup proximally which can lead to non-optimal fixation efficiency.

Another advantage of this present disclosure may include the ability for the user to remove their hand from the handle and maintain the steering position at all times, in addition the user has the means to manipulate the different steering articulation zones independently from each other.

The features disclosed in regard with the system (implantation catheter) may also apply to a method (e.g. for implanting an implant like a leadless pacemaker) and vice versa.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE NUMERALS

1Leadless pacemaker device
10 Housing
100 Distal end
101 Proximal end
11 Electrode
12 Anchoring device
120 Anchoring member
13 Connection member
130 Opening
131 Shaft
132 Rounded face
133 Posts
2 Catheter device
20 Delivery catheter
200 Alignment device
201 Lumen
202 Flush port
203 Tether control
21 Tethering member
210 End
211 Positive-locking member
212 Tether portion
22 Adapter piece
220 Body
221 Retainer groove
222 Recess
223 Slanted face
23 Mandrel
230 Wire clamp
24 Steerable catheter
240 Straight shaft section
241 Proximal steering articulation zone
242 Connection section
243 Distal steering articulation zone
244 Protective cup
245 Guide lumen
246 Guide lumen
247 Flush port
25 Handle device
250-252 Actuation member
27 Steering device (pull wire)
260 Fixing location
Steering device (pull wire)
270 Fixing location
28 Radiopaque element
3 Introducer device
D Distal direction
F Pulling force
L Longitudinal axis
L1, L2 Length
LA Left atrium
LV Left ventricle
M Intra-cardiac tissue (myocardium)
P Proximal direction
R1, R2 Bending radius
RA Right atrium
RV Right ventricle
TV Tricuspid valve

1. A catheter device for implanting a medical devices, comprising:
a steerable catheter, and
a delivery catheter extending through the steerable catheter and being configured to interact with the medical device for implanting the medical device at an implantation site within a patient, wherein the delivery catheter is axially movable with respect to the steerable catheter,
wherein the steerable catheter comprises a first steering articulation zone and a second steering articulation zone arranged distally with respect to the first steering articulation zone, wherein the steerable catheter is steerable by a deflection in the first steering articulation zone and the second steering articulation zone.

2. The catheter device of claim 1, wherein the delivery catheter non-steerable.

3. The catheter device of claim 1, further comprising a steering device which is actuatable for actively causing a deflection in at least one of the first steering articulation zone and the second steering articulation zone.

4. The catheter device of claim 3, the steering device comprises a pull wire.

5. The catheter device of claim 3, further comprising a handle device arranged at a proximal end of the steerable catheter and comprising at least one actuation member configured to control said steering device for causing a deflection in the at least one of the first steering articulation zone and the second steering articulation zone.

6. The catheter device of claim 1. wherein at least one of the first steering articulation zone and the second steering articulation zone is not actively steerable.

7. The catheter device of claim 1, wherein at least one of the first steering articulation zone and the second steering articulation zone is formed by a shape-set curve.

8. The catheter device of claim 1, wherein the first steering articulation zone comprises a first length and/or a first bending radius and the second steering articulation zone comprises a second length and/or a second bending radius, wherein the first length is different than the second length and/or the first bending radius is different than the second bending radius.

9. The catheter device of claim 1, wherein the steerable catheter comprises a straight shaft section arranged proximally of the first steering articulation zone.

10. The catheter device of claim 1, wherein a protective cup arranged on the steerable catheter for sheathing the medical device.

11. The catheter device of claim 1, further comprising:
a mandrel received in an inner lumen of the delivery catheter and movable with respect to the delivery catheter,
an adapter piece connected to the mandrel, wherein the adapter piece, by moving the mandrel with respect to the delivery catheter, is displaceable between a first position in which the adapter piece is received within the inner lumen of the delivery catheter and a second position in which the adapter piece is arranged outside of the inner lumen of the delivery catheter, and
a tethering member comprising a tether portion having a first end at which the tethering member is connected to the adapter piece, wherein the tethering member further comprises a positive-locking member adjoining the tether portion at a distance from the first end,
wherein the positive-locking member in a connection state is received on the adapter piece and is locked with respect to the adapter piece while the adapter piece is in the first position, and
wherein the positive-locking member is releasable from the adapter piece by displacing the adapter piece from the first position towards the second position.

12. The catheter device of claim 11, wherein the positive-locking member comprises a first diameter, measured along a plane perpendicular to a longitudinal axis of extension of the tethering member, larger than a second diameter of the tether portion.

13. The catheter device of claim 11 of 42, wherein the adapter piece comprises a body and a retainer groove formed on the body, wherein the tether portion in the connection state is received in the retainer groove

14. The catheter device of claim 13, wherein the adapter piece comprises a recess formed on the body and adjoining the retainer groove, wherein the positive-locking member in the connection state is received in the recess.

15. An assembly comprising a catheter device of claim 11 and a medical device having a housing and a connection member arranged on the housing, wherein the tether portion of the tethering member in the connection state is connected to the connection member in that the connection member comprises an opening through which the tether portion extends in the connection state.
